SEQUENCE LISTING (8 sequences)

SEQ ID NO: 1 - DNA, 669 bp, Lactobacillus kosoi
  CDS (1)..(669)
  misc_feature (1)..(669): type II-A CRISPR-associated protein Csn2
atgataattagttataagacacataaaaaaataaatatacagcaaggtaaaatcaatgttatcgcaaccaataatcaagctgtatatttagatttcattgatggatttaagggtaatatggacttagttaatatatttgatgataattacaataggctagataatattgaatgtattaattggataggtgatctaattgaatcgattgatattaataaatattataaaaataaattgattaatagtattgataataattttagtgaaaaagaaattaactccattcataatgctaatcgagaattggttaatacaattcaaaagcatttatttatgtacgatctaccaattgaagttgattatgataatgatttaaaaagaattcttaattattcaaaaatccattttgataattatgctatcaataatccttatgataaaataagtatgttaataaaattacatctagaactcgaagattcatcagttttggttgttactaatgtagctcattatttaaccccaaaacaaattgatgggattgcagatttatgtaaaagttcttcaattccattgataatgattgaatttacagatttaaaatctaaggacaattataagcattgtaattttgcatatatagaccaagatttcgttgattggtattaa

SEQ ID NO: 2 - protein, 222 aa, Lactobacillus kosoi
MIISYKTHKKINIQQGKINVIATNNQAVYLDFIDGFKGNMDLVNIFDDNYNRLDNIECINWIGDLIESIDINKYYKNKLINSIDNNFSEKEINSIHNANRELVNTIQKHLFMYDLPIEVDYDNDLKRILNYSKIHFDNYAINNPYDKISMLIKLHLELEDSSVLVVTNVAHYLTPKQIDGIADLCKSSSIPLIMIEFTDLKSKDNYKHCNFAYIDQDFVDWY

SEQ ID NO: 3 - DNA, 306 bp, Lactobacillus kosoi
  CDS (1)..(306)
  misc_feature (1)..(306): CRISPR-associated endonuclease Cas2
atgcgcttaatagtaatgtttgatttaccaactgatactagtgaagatcgaagaaattatcgtcattttcgtaaaaaattaattaatgaaggttttatgatgttacagtattctgtttatgaaagagtttgtgttaacaggaagtcagctagatttttggaaaatagaattgctgcgatggcaccagataaaggccttattcaaagctttatgattactgaaaagcaatataatgatattcattttattaccgggaagccgattgaagacgtccgtaattcatcagaaaggaccattattctatga

SEQ ID NO: 4 - protein, 101 aa, Lactobacillus kosoi
MRLIVMFDLPTDTSEDRRNYRHFRKKLINEGFMMLQYSVYERVCVNRKSARFLENRIAAMAPDKGLIQSFMITEKQYNDIHFITGKPIEDVRNSSERTIIL

SEQ ID NO: 5 - DNA, 903 bp, Lactobacillus kosoi
  CDS (1)..(903)
  misc_feature (1)..(903): type II CRISPR-associated endonuclease Cas1
atgggatggcgaaatgtaattatttctcagcatgctaagttatcttattcagctaataatatgattgtccaaacttttgaaggtattaatcagatacctatttctgatatgtatattttgattattgaaagtaatcaagcagttataacaactgagttaataagccagttaaattttcatgatgtaaaaattgttttttcagatgacaatcacaatccttgttgtgagacagtaaactattatccaaataatcgttcgcttgataaattagaaaaacaatacaattggtcaagcaatcgtgtaaatattttatggacaaaaataattaataataaaattataaatcagattaatgttttaaaattttataatcataatactgaaactttagaaaatgaattaagtaagttagaacttggtgatgttagtaatcgcgaagcagtagtggcaagaaaatattttatgctgttatttgataaaaaattctcacgacgtgatttttcaccaatgaatgctgctttaaattatggttattctattttattatcaacctttaatcgctcaattgtcttgaatggatatttaactgagattggaattcatcaccatagtgatgaaaataattttaatttaggatcagattttatggagccctttagaccaattgttgattattggttagctaataagaaatttaatcaactgacaccagatataaaatttagtttagtagaattattaaatgttgaaataaaatataatggcaaaaatatggttttaaataatgcaatagataaatatacagatgattgtttgaattacttaactaccggaaaatctataaagatagaggttgaattgataaatgaggtaccgaataatgcgcttaatagtaatgtttga

SEQ ID NO: 6 - protein, 300 aa, Lactobacillus kosoi
MGWRNVIISQHAKLSYSANNMIVQTFEGINQIPISDMYILIIESNQAVITTELISQLNFHDVKIVFSDDNHNPCCETVNYYPNNRSLDKLEKQYNWSSNRVNILWTKIINNKIINQINVLKFYNHNTETLENELSKLELGDVSNREAVVARKYFMLLFDKKFSRRDFSPMNAALNYGYSILLSTFNRSIVLNGYLTEIGIHHHSDENNFNLGSDFMEPFRPIVDYWLANKKFNQLTPDIKFSLVELLNVEIKYNGKNMVLNNAIDKYTDDCLNYLTTGKSIKIEVELINEVPNNALNSNV

SEQ ID NO: 7 - DNA, 4377 bp, Lactobacillus kosoi
  CDS (1)..(4377)
  misc_feature (1)..(4377): type II CRISPR-associated endonuclease Cas9
atgaaaaaatatcatattggtttagatataggaacatcatcagtaggatttgctgctaagcatgatgatggtagtttggttcatgtaaagggtaaaaatgttattggtgctcgtttatttaatgaaggccaaactgcagaagaacgccgtacatatagagctgctagaagaagatatagtcgcagaagatggagattaaacttattaaatcaaatttttaaagattctttagatgcagtagatgctacttttctaaaaaggcttaaagaatctagcttatctaatcaagatcaaaataaaaaatattttggtgaactattattccccaaagataatgataaacattttcatacaaacaatattcaagaaaataaacgtggtaatgatactatttttcatttacgtgatcgattaatgaaatcaaatgaaaaagcagatattagggagatttatttagcttttcattcgatggtaaaaaatcgtggtaactttttggataataccccagcatcttcttttgaagcatcggatatgcatttagtagatgtgttacataatataaataatctttatgaacaaatgaatattacttttggtcttaatgatgtaaatgctgaaaaaattgaagatattttattggacaataaagtgcgtaatattgataagaaaaaaacattaattaatttattacaaactaaagaaagtaataaagaatctaaaaccattattactgaaattgtgaaattgattttaggatataatagcaaaaaaattaatgccattttagaagtagaagaactagaaaataatgaattatgtttatctaatgctaatagcgatgatcaaattaatgcaatctttgctgaaacaaatgaagttcaacaaaatattattactgaaattaagactttatattcaagaaccaagttaaatcaaattatccctaatggtaaaacttattctgaatcgatgattgataagtataatttacatcatgatcaattacgtaatttaaaactgaatattttagaccatttaaactttgataaaaacagaaagaataatttaaaaatagcttatgctgcctatgttggtaatttagataaagaaaatttttcatctgatgatttgaatttatttatagacagtattaataacaaagacggtaaaggtgctattaaaaaagttataaatggccataagaagaaaattacacaaagtgatttatttactatttttcatgatctacttggaaatcctgaactaactgatactttaagtaatttattatcgtctgttcctatgtctttaaaagattttgaaaatgaaacgaaagaattaatgaacaatgatagttattctgtagttcaaaaagatattaaagcattaaaaaagggtaaaaagaaagatgatcttattgtaaaagatgatattcaacaaattttaaataataacccactaaaagacttgttcaatcatgaaactatcaaaaaagaaattattaaaattaataaatcaattcaattgtgcaattatttaccaaaacttagaacttcagataatgccagcattccacatcaaattaatcaaaatgaaatgaatcaaattattgaaaagcaaaagcaatattatccttggttgtcggaaccaaatcctaattcaaatcgcaaaaatattgccaagtataaactagatgaattaattgcttttagaattccatattatgttggaccaatgattacgtctgatgatcaacaaaatagttctaatgctaattttgcatggatgaagagaaaagctagtggcgtgattacaccatggaattttgaagataaggttgatgtaaaatctactgctaccgaatttattaagcgaatgactgttaaagatacttatttaattaatgaagatgtattaccagataatagtttgttatatcaagaatttaaggtattaaacgaactaaatatcgtaaaagcgaatggaaaacacttaacagtcgaacaaaaacaatccgtttttaatgatttatttaaaaagcaaaaaacggtgactgctaagaaattaagtaattatttagctaatgactatgtaacaccacccgaaataactggactttcagataaagaaaagtttaataataattatggttcatatattgatatgaagaaaatatttggtgataagattgatgatttgagcttgcgtgatgattttgaaaagatgattgaatggtcaactatatttgaagatcgagatattttaagattacaacttaaagatgttaagtggttaaataaaaatcagattaaccaattaattagtaagcgttatagtggttggggtcgattatcaaaaagattattgatggggttattaaatgacaatggtgaaagaattattgacgttctatggaatactccagctaattttatgcaagcagtaaataatccagatattaaaacacaaattgcaaaaattaattcgaaacaggtaaataacttaggtatggaagcaattttagataatgcctacacctcaccacaaaataaaaaagcaattagacaagcaatcaaagtagttaatgatattcaaagagctatgaagggtcaagcaccagcatccatatcaattgaattcactcgaaaacctgaaaacaattccgatattactaaaagtcgtggtaaacagatagataaaatatataaggaattatctaataacatatctaaagatttaaaagatgaattaaaaactaacaagaagaatctatctgataaattatatctatatttcatgcaaaaaggacgagatatttataccggtgaatctatagatattgataatttgattaattatgatattgaccatattattccaagaagttatatgaaggatgattcatttaataatcgtgttcttactaaccatcaaactaatgatgataagggtgataaaacaccattagatggactatcatcgattaatattaataatcaaattccagaatggaaaaaattattaaaacagggacttattagtgttcgtaaattccgtaatttaactactaagatagatgcaattagtaagtatactaaaaatggatttgtacatcgtcaattagttgaaaccagtcaggttattaaattagttgctaatattctaaataataaataccaaaatagtgataccaatattattgaagttaaagcttatatgaatactcaattaagagaaacatttgacttatttaagtccagagaattgaatgattatcatcatgctttagatgcatatttaacaacttttgcaggaacttatttatatgatcgttatcctaaattaagacattattttgtatatggtaattttaaaaagttcgatgattcaaaaactattaatcatttaaaaacttttaattttctacgtgatattaccagtccaagtaaagaacatgaagataaaatatttgataaagctagtggtgaattaattttaaatcgtaaaaaagccattaatcgaattaagcaaatttataattataaatatatgttagttactcatgaagtttctactagaaaaaacgctttatataatcaaagtatttatcctgctaaaaatgttaagaagagttttattaacattaaaaatgataaaccagttgaattatacggtggacatactggtaacaataatgcttatatggctttagtcaaaatcattggtaagaatggaaatgaatataaattagttggcgtgccaattagattcttgtctgacttaaatagggctaaaaaggataacttgtctgcttatcaaaataagttacatgaaatgattagtgcacaactgcctaataaaaagtttacagttcttttagataaagttatgtatcgccaattagttgttgatggggatgaaaaatatacagttggcagtgctacttataaatataatgctaagcaactggttatttctccaaaatcagttagaattttgaaagataaagaattaagaaatagtctatcatccaaacaattaagtgagaaattaaattttgtatataatgatatattgaaacaagttaatagatatttaccactgtatgatattaattcatttaggaataaattaaatgctggttttgataagtttaaaaatattagtgataattttgaaaaaacgcaaattttaaataatattttggaaggacttcacgataatccaagacaaattgcaattaagaaaataggatttacaacgccttttggaatgatgcaattaaataatggaattaaactatctaaaaattcatatattatttatcaatctccaaccggattatttgaacgaaaagttagaattaaagacttataa

SEQ ID NO: 8 - protein, 1458 aa, Lactobacillus kosoi
MKKYHIGLDIGTSSVGFAAKHDDGSLVHVKGKNVIGARLFNEGQTAEERRTYRAARRRYSRRRWRLNLLNQIFKDSLDAVDATFLKRLKESSLSNQDQNKKYFGELLFPKDNDKHFHTNNIQENKRGNDTIFHLRDRLMKSNEKADIREIYLAFHSMVKNRGNFLDNTPASSFEASDMHLVDVLHNINNLYEQMNITFGLNDVNAEKIEDILLDNKVRNIDKKKTLINLLQTKESNKESKTIITEIVKLILGYNSKKINAILEVEELENNELCLSNANSDDQINAIFAETNEVQQNIITEIKTLYSRTKLNQIIPNGKTYSESMIDKYNLHHDQLRNLKLNILDHLNFDKNRKNNLKIAYAAYVGNLDKENFSSDDLNLFIDSINNKDGKGAIKKVINGHKKKITQSDLFTIFHDLLGNPELTDTLSNLLSSVPMSLKDFENETKELMNNDSYSVVQKDIKALKKGKKKDDLIVKDDIQQILNNNPLKDLFNHETIKKEIIKINKSIQLCNYLPKLRTSDNASIPHQINQNEMNQIIEKQKQYYPWLSEPNPNSNRKNIAKYKLDELIAFRIPYYVGPMITSDDQQNSSNANFAWMKRKASGVITPWNFEDKVDVKSTATEFIKRMTVKDTYLINEDVLPDNSLLYQEFKVLNELNIVKANGKHLTVEQKQSVFNDLFKKQKTVTAKKLSNYLANDYVTPPEITGLSDKEKFNNNYGSYIDMKKIFGDKIDDLSLRDDFEKMIEWSTIFEDRDILRLQLKDVKWLNKNQINQLISKRYSGWGRLSKRLLMGLLNDNGERIIDVLWNTPANFMQAVNNPDIKTQIAKINSKQVNNLGMEAILDNAYTSPQNKKAIRQAIKVVNDIQRAMKGQAPASISIEFTRKPENNSDITKSRGKQIDKIYKELSNNISKDLKDELKTNKKNLSDKLYLYFMQKGRDIYTGESIDIDNLINYDIDHIIPRSYMKDDSFNNRVLTNHQTNDDKGDKTPLDGLSSININNQIPEWKKLLKQGLISVRKFRNLTTKIDAISKYTKNGFVHRQLVETSQVIKLVANILNNKYQNSDTNIIEVKAYMNTQLRETFDLFKSRELNDYHHALDAYLTTFAGTYLYDRYPKLRHYFVYGNFKKFDDSKTINHLKTFNFLRDITSPSKEHEDKIFDKASGELILNRKKAINRIKQIYNYKYMLVTHEVSTRKNALYNQSIYPAKNVKKSFINIKNDKPVELYGGHTGNNNAYMALVKIIGKNGNEYKLVGVPIRFLSDLNRAKKDNLSAYQNKLHEMISAQLPNKKFTVLLDKVMYRQLVVDGDEKYTVGSATYKYNAKQLVISPKSVRILKDKELRNSLSSKQLSEKLNFVYNDILKQVNRYLPLYDINSFRNKLNAGFDKFKNISDNFEKTQILNNILEGLHDNPRQIAIKKIGFTTPFGMMQLNNGIKLSKNSYIIYQSPTGLFERKVRIKDL

(19) United States
(12) Patent Application Publication
(10) Pub. No.: US 2020/0179466 A1
(43) Pub. Date: Jun. 11, 2020

(54) Novel Bacterial Strain Of Lactobacillus And Immunostimulant Comprising The Same

(71) Applicant: AOB KEIOH GROUP CORP., Hokuto-shi (JP)

(72) Inventors: Tai-Ying CHIOU, Kitami (JP); Wataru SUDA, Yokohama (JP); Kenshiro OSHIMA, Tsukuba (JP); Masahira HATTORI, Shinjuku-Ku (JP); Chiaki MATSUZAKI, Nonoichi (JP); Kenji YAMAMOTO, Otsu (JP); Tomoya TAKAHASHI, Hokuto-Shi (JP)

(21) Appl. No.: 16/429,483

(22) Filed: Jun. 3, 2019

(30) Foreign Application Priority Data
Dec. 10, 2018 (JP) 2018-230534

(51) Int. Cl. A61K 35/747; C12N 1/20; C12R 1/225; A23L 33/135; A23K 10/16

(52) U.S. Cl. CPC A61K 35/747; C12N 1/20; A23V 2200/324; A23L 33/135; A23K 10/16; C12R 1/225

(57) ABSTRACT

Based on the microbiological and biochemical characteristics of a novel strain of lactic acid bacteria emerged in the fermentation process of a vegetable and brown sugar fermentation beverage, a better immunostimulant and a health care food, a pharmaceutical composition and the like including the immunostimulant are provided. Disclosed are an isolated bacterial strain of Lactobacillus kosoi 10H deposited under the accession number NITE BP-02811, and a composition comprising the isolated bacterial strain or a culture product thereof.

### CROSS-REFERENCE TO RELATED APPLICATIONS

This application claims priority based on Japanese Patent Application No. 2018-230534 filed on Dec. 10, 2018 in Japan, and all contents described in the application are hereby incorporated by reference in their entirety. In addition, all contents described in all patents, patent applications, and documents cited in the present application are incorporated herein by reference in their entirety.

### TECHNICAL FIELD

The present invention relates to a novel bacterial strain of lactobacillus kosoi 10H, an immunostimulant comprising the same, and the like.

### BACKGROUND ART

A health beverage comprising an enzyme solution as its main ingredient, made by fermenting and ripening raw materials such as vegetables, fruits and seaweeds, is known to enhance immunity and to be effective for adjusting physical condition. A vegetable and brown sugar fermentation beverage (for example, trade name “Georina® enzyme”) is an enzyme drink obtained by natural fermentation of the raw materials such as unrefined sugar, leaf vegetable, root vegetable, brown sugar, galacto-oligosaccharides, stem vegetables, potatoes, mushrooms, flower vegetables, Kombu and seaweed etc., by complex population of microorganisms. The microbiota appeared and disappeared in the fermentation process of this vegetable and brown sugar fermentation beverage is reported to have such features that lactic acid bacteria become predominant in a short period of time by natural fermentation, the pattern of microbiota in each batch is stable, and the bacterial community in the fermentation beverage dramatically changes, resulting in significantly low diversity at the end of fermentation as compared with the complex starting community. (for example, see Non-Patent Literature 1).

Meanwhile, various studies have been conducted on the immunomodulatory function of lactic acid bacteria. For example, it is known that lactic acid bacteria belonging to the genus Lactobacillus of the plant origin have a function to activate intestinal tract immunity and to promote immunoglobulin A (IgA) antibody production in Peyer's patch cells (Patent Literature 1), lactic acid bacteria belonging to the genus Leuconostoc have an activity of stimulating immunity (particularly intestinal tract immunity) (Patent Literature 2), and the lactic acid bacteria of Lactobacillus brevis and Lactobacillus casei separated from Narezushi of horse mackerel has an anti-allergic effect (Patent Literature 3) and the like.

As mentioned above, certain lactic acid bacteria activate acquired immune system of the host and promote secretion of IgA into the intestinal tract, whereas the immunostimulatory activity of these bacteria, even though species is identical, differs greatly when the strain is different. Its activation mechanism is not completely clear, but it is believed that the intestinal-related lymphoid tissue called gut-associated lymphatic tissue (GALT), in particular, Peyer's patches produce IgA, which is secreted into the intestinal tract, binds to harmful bacteria and viruses to hinder their movement, and inhibits their adhesion to intestinal epithelial cells. In addition, the following mechanism has also been proposed that the microbiota stimulates intestinal epithelial cells via the toll-like receptor (TLR) and dendritic cells (DCs) to secrete various cytokines, which induces the differentiation of IgA-producing B cells present in the mucosal lamina propria (see, for example, Non-patent Literature 2, FIG. 1).

### CITATION LIST

### Non-Patent Literature

Non-Patent Literature 1: Chiou T-Y, Suda W, Oshima K, Hattori M, Takahashi T (2017) Changes in the bacterial community in the fermentation process of koso, a Japanese sugar-vegetable fermented beverage. Biosci Biotechnol Biochem 81(2):403-410.
Non-Patent Literature 2: Kamada N. et al., Role of the gut microbiota in immunity and inflammatory disease. Nat Rev Immunol. 2013 May; 13(5):321-35.

### Patent Literature

Patent Literature 1: Japanese Patent Laid-Open No. 2007-308419
Patent Literature 2: WO2014/129599
Patent Literature 3: Japanese Patent Laid-Open No. 2013-193996

### SUMMARY OF THE INVENTION

### Technical Problem

The vegetable and brown sugar fermentation beverage is known to have a function to promote health by ingesting as a health drink, but its active ingredient is not completely clear. The present invention has been made under such circumstances, and an object thereof is to clarify the microbiological and biochemical characteristics of lactic acid bacteria appearing in the fermentation process of vegetable brown sugar fermentation beverage, and to provide a better immunostimulant as well as a health food, a pharmaceutical composition and the like containing the immunostimulant.

### Solution to Problem

As a result of analyzing the change of microbiota during the fermentation process of the vegetable brown sugar fermentation beverage based on the number of readings of the 16S rRNA gene sequence using the next generation sequencer, it was found that in the culture broth after the seven day fermentation, a novel lactic acid bacterial species belonging to the genus Lactobacillus becomes predominant, and accounts for 50% or more of the microbiota. Finally, the present invention has been completed by isolating the novel lactic acid bacterium.

That is, in one embodiment of the present invention, there is provided an isolated bacterial strain of Lactobacillus kosoi 10H deposited under accession number NITE BP-02811. The strain preferably comprises nucleotide sequences of SEQ ID Nos. 1, 3, 5 and 7.

Another embodiment of the invention is a method for stimulating immunity of a subject in need thereof, comprising administering to the subject a composition comprising an effective amount of cells of Lactobacillus kosoi 10H deposited under accession number NITE BP-02811. The immunity is stimulated preferably by promoting IgA production in mucosa of the subject. The mucosa may be intestinal mucosa and the subject may be mammals. More preferably, the cells are heat-treated at 70° C. for one minute or more. The composition is used in a form of a food and drink, a pharmaceutical composition, an external preparation or a feed.

In yet another embodiment, there is provided an IgA production promotor or an immunostimulant comprising the isolated bacterial strain of Lactobacillus kosoi 10H, as an active ingredient, the strain being deposited under accession number NITE BP-02811. The IgA production promotor or the immunostimulant are used in a form of a food and drink, a pharmaceutical composition, an external preparation or a feed.

In another aspect of the present invention, there is provided a process for producing a composition for stimulating immunity of a subject in need thereof, which process comprises the steps of inoculating the isolated bacterial strain of Lactobacillus kosoi 10H deposited under the accession number NITE BP-02811 in a culture medium of high sugar concentration, wherein the sugar includes D-fructose, and growing cells of the bacterial strain in the culture medium.

### Advantageous Effects of Invention

The novel lactic acid bacterial strain of the present invention has an excellent IgA production promoting effect as compared with existing lactic acid bacteria and bifidobacteria and is useful for activating mucosal immunity of the intestinal tract and the like.

### BRIEF DESCRIPTION OF DRAWINGS

FIG. 1 shows growth characteristics of strain 10H cultured in MRS broth supplemented with additives of various concentrations.

FIG. 2 shows a phylogenetic tree based on 16S rRNA gene sequences.

FIG. 3 shows a result of measurement of IgA production inducing activities of 16 samples of lactic acid bacteria including strain 10H and bifidobacteria.

FIG. 4A shows IgA production inducing activities of samples of lactic acid bacteria and bifidobacteria treated at 70° C. for 30 minutes.

FIG. 4B shows IgA production inducing activities of samples of lactic acid bacteria and bifidobacteria treated at 100° C. for 30 minutes.

### DESCRIPTION OF EMBODIMENTS

Hereinafter, preferred embodiments of the present invention will be described in the following order.

(I) Novel lactic acid bacterium
(II) Immunostimulatory action
(III) Various compositions and uses thereof
(IV) Process for producing the immunostimulant

### (I) Novel Lactic Acid Bacterium

According to a preferred embodiment of the present invention, a novel lactic acid bacterial strain isolated from vegetable brown sugar fermentation broth and mutants thereof are provided. More preferably, the strain is a bacterium belonging to the genus Lactobacillus obtained from a culture broth of a product, the trade name “Georina® enzyme”. Still more preferably, the strain is Lactobacillus kosoi 10H strain (Accession number NITE BP-02811) or a mutant strain thereof. The term, “mutant strain” refers to a mutant of a specific strain prepared by those skilled in the art with a well-known method, to the extent that its main properties are not changed, or the mutant strain includes strains which can be recognized by those skilled in the art as being equivalent to the specific strain.

In addition, the strain of Lactobacillus kosoi 10H has been deposited on Nov. 7, 2018 (date of the original deposit), at NITE Patent Microorganisms Depositary, National Institute of Technology and Evaluation (#122, 2-5-8 Kazusakamatari, Kisarazu-shi, Chiba 292-0818, Japan). The accession number is NITE BP-02811 (hereinafter this strain is referred to as “strain 10H”).

(Phenotypic Characteristics of Strain 10H)

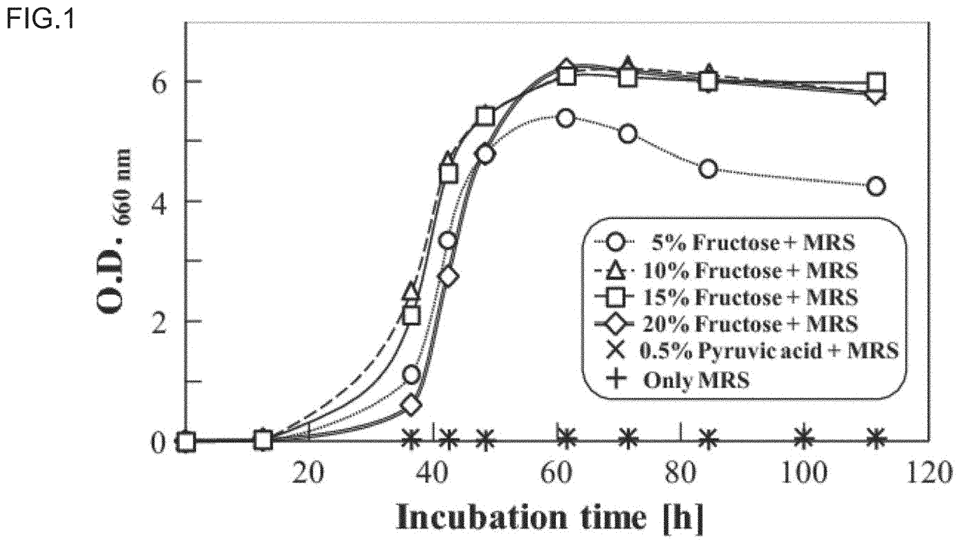

Strain 10H is a Gram-positive, catalase negative and rod-shaped strain, that shows a morphology of approximately 0.7-0.8×1.5-2.2 μm. The fructophilic growth characteristics of this strain are shown in FIG. 1. Strain 10H does not grow in only MRS broth unless D-fructose is supplemented and it is observed to grow well even when the concentration of D-fructose was increased up to 20%. Colonies are observed to form on MRS agar supplemented with 5-10% D-fructose but very few or no colonies are observed on media with 4% D-fructose or below. This characteristic explains why this strain could not be isolated so far by conventional methods as a difficult-to-culture species. Colonies are observed to form under aerobic, microaerophilic and anaerobic conditions. They appear white and opaque, circular and entire, raised and moist, with a diameter of approximately 1-3 mm. Other microbiological characteristics are as follows.

Growth pH: 4.0-7.0 (Optimum pH is 6.5)

Growth temperature: 18-39° C. (optimum temperature is 27° C.)

### Production of gas: yes

NaCl resistance: growth suppression at 2% (w/v) or more

### Sugar Assimilation:

Carbon-source utilisation were examined using API 50CHL according to the manufacturer's instructions (bioMérieux). The results, however, showed no utilisation with all carbohydrates in the analysis of API 50CHL at 30° C. Therefore, carbohydrate utilisation was further investigated using 20 kinds of carbohydrate (D-galactose, L-sorbose, D-xylose, D-mannose, L-rhamnose, D-arabinose, L-arabinose, trehalose, maltose, lactose, melibiose, D-cellobiose, D-raffinose, D-melezitose, inulin, D-ribose, D-lyxose, D-fructose, galactooligosaccharide, sucrose) in MRS broth and cultured at 27° C. for 48 hours. According to the results of HPLC analysis, using 20 carbohydrates, only D-fructose and sucrose were observed to be utilised after 48-h incubation. The results of metabolite analysis showed that the strain 10H utilises D-fructose and produces lactic acid and acetic acid but no ethanol. The ratio of lactic acid to acetic acid was approximately 3:2.

### Enzyme Activity:

Enzyme activity was examined using API ZYM test strips according to the manufacturer's instructions (bioMérieux). Strain 10H was observed to produce activities of acid phosphatase and naphthol AS-BI-phosphohydrolase, and to be weakly positive for the actions of alkaline phosphatase, lipase, leucine arylamidase and valine arylamidase.

### (Chemotaxonomic Characteristics)

The amino acid composition of cell wall of strain 10H was identified as predominantly aspartic acid (Asp), glutamic acid (Glu), alanine (Ala), and lysine (Lys). Neither meso-di-aminopimelic acid nor ornithine were found to be present in the cell wall peptidoglycan, and the molar ratio of Asp/Glu/Ala/Lys was 1.0/1.9/4.1/1.1. This suggests the cell wall peptidoglycan of strain 10H to be the A4a L-Lys-D-Asp type described by Schleifer and Kandler (1972, Bacteriol Rev 36:407-477), which is the major peptidoglycan type reported in the literature describing members of Lactobacillus (Schillinger U, Lucke F K (1987) Food Microbiol 4:199-208).

The major fatty acids detected in strain 10H were identified as C16:0 (37.6%), C19:0 cyclopropane 11,12 (28.7%), C19:0 cyclopropane 9,10 (14.3%) and C18:1ω9c (10.0%). The major components of the polar lipid in strain 10H by two-dimensional high-performance thin layer chromatography were lysophosphatidylethanolamine (LPE), phosphatidylethanolamine (LPE) and glycolipid. Generally, it is said that the Lactobacillus group is deficient in isoprenoid quinone, but from strain 10H, an extremely small amount of isoprenoid quinone was found to be 0.0001 to 0.0004 nmol/g of dry weight. The main quinone detected in the 10H strain was menaquinone (MK), and ubiquinone and plastoquinone were not detected. Major isoprenoid quinones detected in strain 10H are MK-7, MK-8, MK-9 and MK-10.

### (Phylogenetic Analysis)

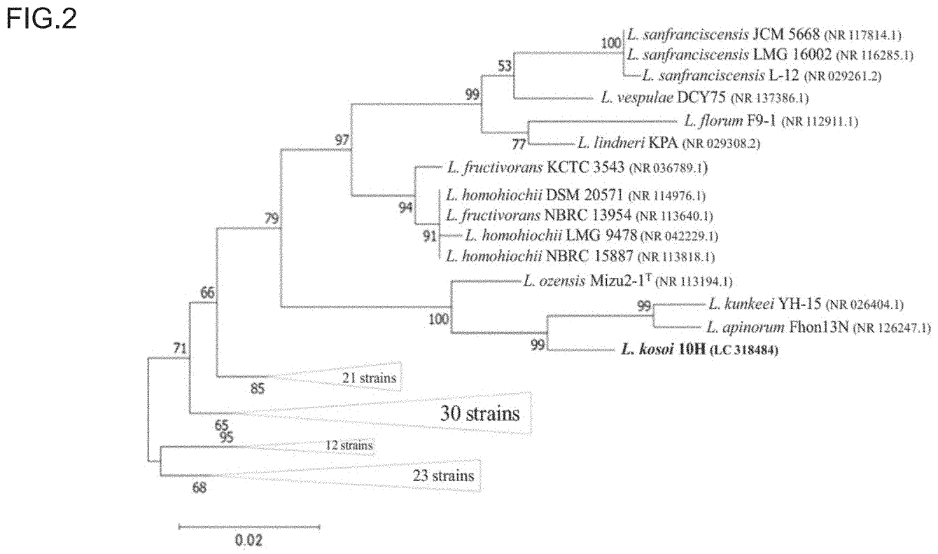

The 16S rRNA gene sequence of strain 10H is a continuous stretch of 1474 bp (GenBank/EMBL/DDBJ accession number LC318484). According to the nucleotide BLAST results based on 16S rRNA gene sequences, the most similar three type strains to strain 10H were identified as L. kunkeei YH-15, L. ozensis Mizu2-1 and L. apinorum Fhon13N with sequence similarities of 95.5% (66 nucleotide (nt) differences at 1452 sites), 95.4% (67 nt differences at 1403 sites), and 95.3% (68 nt differences at 1456 sites), respectively. These sequences similarities are significantly lower than the 98.65% threshold recommended for prokaryotic species differentiation (Kim M, Oh H S, Park S C, Chun J (2014) Int J Syst Evol Microbiol 64:346-351). According to the phylogenetic tree prepared using the maximum-likelihood method, strain 10H forms one distinct phylogenetic cluster with the three closely related species (see FIG. 2).

In the draft genome of strain 10H analyzed in Example 3, hexokinase (EC 2.7.1.1) is not found and a fructokinase (EC 2.7.1.4) is annotated instead. This result may explain why strain 10H grows better in fructose-containing MRS broth rather than glucose and other hexoses. In addition, since the gene of sucrose-6-phosphate hydrolase (EC 3.2.1.26) is present in the draft genome, it is assumed that strain 10H can be used as sucrose as an energy source. These results explain that strain 10H grows well and even becomes the dominant bacterial species in a fermentation broth containing high concentration sucrose of 40% by mass or more.

Furthermore, the presence of at least 4 genes associated with the CRISPR-Cas system is estimated in the genome of strain 10H. Specifically, the type II-A CRISPR related proteins Csn2 (SEQ ID NOs: 1 and 2), CRISPR related endonucleases Cas2 (SEQ ID NOs: 3 and 4), type II CRISPR related endonucleases Cas1 (SEQ ID NOs: 5 and 6) and type II CRISPR RNA-derived endonuclease Cas9 (SEQ ID NOS: 7 and 8). Sequence numbers 1 to 8 of the Sequence Listing indicate the DNA sequence encoding these proteins and the deduced amino acid sequence. The CRISPR-Cas system is important as a bacterial immune system and has possibility to be used as a genome editing tool. In addition, the double-stranded RNA synthesized by these systems may be involved in TLR3-mediated innate immunostimulatory action in living organisms ingesting the strain 10H.

Based on the above characteristic analysis and taxonomic analysis, strain 10H is concluded to represent a novel species, which is deposited as the name Lactobacillus kosoi, under the Center Biological Genetic Resource Deposit System (Accession Number: NBRC 113063) of National Institute of Technology and Evaluation. Thereafter, the strain NBRC 113063 was transferred to the Patent Organism Depositary Center and deposited as an accession number NITE BP-02811. The taxonomic properties of the novel lactic acid bacterium described in this specification are also described in a paper published by the present inventors (Chiou T Y et al, Lactobacillus kosoi sp. nov., a fructophilic species isolated from koso, a Japanese sugar-vegetable fermented beverage. Antonie van Leeuwenhoek (2018), 111: 1149-1156), the entirety of which is hereby incorporated by reference.

### (II) Immunostimulatory Action

In the present specification, the term, “immunostimulant” refers to a composition containing, as an active ingredient, cells of Lactobacillus kosoi strain 10H (Accession No. NITE BP-02811) or a culture product thereof. The immunostimulant promotes secretion of IgA in mucosal epithelia of an oral cavity, nasal cavity, respiratory organ, gastrointestinal tract, etc. and is effective for activating immune system of a subject. However, the mechanism of immunostimulatory action is not limited to the promotion of IgA secretion, but may include other known or unknown mechanisms. As described in detail below, the immunostimulant of the present invention includes a form of a food and drink, a pharmaceutical composition, an external preparation and/or a feed. Among them, a health food is preferable, and particularly preferable is a food composition for maintaining and promoting health of a subject in need thereof, i.e. a subject with reduced immunity.

The term, “IgA production promotor” refers to a composition containing cells of Lactobacillus kosoi strain 10H (Accession No. NITE BP-02811) or a culture product thereof, as an active ingredient, which has an ability to enhance IgA production in mucosa of the subject. The enhancement of IgA production can be shown by culturing Peyer's patch cells containing a large amount of IgA production cells with the composition for a predetermined period of time, and measuring an increase of the amount of secreted IgA into the culture solution compared to the case without addition of the composition. By administering the IgA production promotor together with a vaccine, it would be possible to enhance the production of antibodies corresponding to antigens contained in the vaccine, to enhance the effect of the vaccine, and to suppress the side effects of the vaccine. That is, the IgA production promotor enhances the production of antibodies against the antigen contained in the vaccine and enhances the induction of protective immunity to increase the effect of the vaccine.

### (III) Various Compositions

When the novel lactic acid bacterium of the present embodiment is used in the form of various compositions such as a food and drink, pharmaceutical composition, external preparation (topical medicines, cosmetics etc.), feed and the like, the bacterial cells of the lactic acid bacterium are cultured according to conventional methods of culturing lactic acid bacteria. From the obtained culture media, the bacterial cells separated by centrifugation or the like can be used as they are, as well as a culture/fermentation solution (culture supernatant), a crude or purified product of the culture, a lyophilized product or a fraction of cytoplasm or cell wall obtained by treating the bacterial cells with an enzyme or physical means can also be used.

In addition, the bacterial cells may be not only viable cells but also sterilized cells by ordinary general heat sterilization operation. An immunity inducing activity of lactic acid bacteria derived from protein components, nucleic acids and the like, which are susceptible to heat denaturation, is generally reduced by heat treatment at 70° C. for 30 minutes. In contrast, as is clear from the results of Example 4 described later, the IgA production-inducing activity of strain 10H is not attenuated by heat-treatment, that is, the immunity-enhancing component produced by strain 10H has heat resistance. Because strain 10H is capable of growing under severe growth conditions such as high sugar concentration (high osmotic pressure), the strain is expected to have a robust cell surface structure. The temperature of the heat treatment is preferably at least 70° C., for example, at 70° C., 75° C., 85° C. or more. The time of the heat treatment is at least one minute, for example, 1, 2, 5, 10, 30 minutes or more. The temperature and time for the heat treatment can be adjusted by appropriate combination, and a typical condition of the heat treatment is at 70° C. for one minute or more. Since heat-treated cells are expected to have a sufficient immunostimulatory action such as induction of IgA production etc., while viable cells may cause a morphological change at the time of delivery or display after product manufacturing, the heat sterilized cells that do not cause any further change in morphology can be preferably used. When strain 10H is contained as sterilized cells in the composition of the present embodiment, conditions for the sterilization such as heating, pressurization and the like may be adopted for each of the commercialized compositions.

For example, as shown in Example 1, the culture broth can be obtained by culturing at 18 to 39° C. for about to 28 hours using a suitable medium for lactic acid bacteria of the present embodiment, for example, MRS medium containing D-fructose and the like. The cultured cells can be obtained after culturing, for example, by centrifuging the culture solution at 3,000 rpm, 4° C. for 10 minutes, and collecting the cells. These cells can be purified according to a conventional method. Furthermore, the cells can be lyophilized or spray dried. The bacterial cells thus obtained can be used as an active ingredient of the composition of the present embodiment.

In the composition of the present embodiment, the bacterial cells of strain 10H can be used as such, but it is also possible to properly mix with a suitable edible carrier (food material) and a pharmaceutically acceptable carrier to prepare foods and drinks, pharmaceutical compositions, external preparations, feeds and the like.

In addition, in the composition of the present embodiment, an appropriate amount of nutrient components suitable for maintaining or growing strain 10H can be further contained, if necessary. Specific examples of such nutrient components include carbon sources such as glucose, starch, sucrose, lactose, dextrin, sorbitol, fructose and the like, which are used as culture media for culturing microorganisms, nitrogen sources such as yeast extract, peptone and the like, vitamins, minerals, trace metal elements, other nutritional components, and the like. Examples of vitamins include vitamin B, vitamin D, vitamin C, vitamin E, vitamin K, and the like. Examples of trace metal elements include zinc, selenium and the like. Examples of other nutrients include various oligosaccharides such as lactosucrose, soybean oligosaccharide, lactulose, lactitol, fructo-oligosaccharide, galacto-oligosaccharide and the like. The blending amount of these oligosaccharides is not particularly limited, but it is usually preferably selected from the range of 1 to 30% by weight in the composition of the present invention.

In general, the blending amount of strain 10H in the composition of the present embodiment can be those appropriately selected from the number of bacteria from 10⁸ to 10¹¹ (unnecessary to be viable) in 100 g of the composition. The number of viable bacteria is determined by applying a diluted sample to an agar plate for bacterial culture, culturing at 30° C., and measuring the number of grown colonies. Since this viable cell number correlates with turbidity of culture broth, if the correlation between viable cell numbers and turbidities is previously determined, the viable cell number can be counted by measuring the turbidity instead of the viable cell counts. The blending amount of strain 10H can be appropriately changed depending on the form of the composition of the present embodiment to be prepared with the above amount as a guide.

The composition of the present embodiment is used together with the vaccine or the composition alone. When used together with a vaccine, the composition can be administered before or after administration of the vaccine, and can also be used as a potentiating agent for a vaccine which enhances the effect. The use amount of the composition varies depending on the type and quality of the vaccine used, age, symptom, etc. For example, for use in prevention, about 0.01 to 10 g in terms of solid content per adult is preferable to take it three times a day for 30 minutes before meals. In addition, when used as a health food, it is preferable to add an amount that does not adversely affect the taste or appearance of the food, for example, within the range of about 0.1 to 100 g in terms of solids based on 1 kg of the target food.

The form of each composition will be specifically described below.

### (Food and Drink)

When the composition of the present embodiment is used as a food and drink, for example, fermented milk, lactic fermenting beverage, fermented vegetable drink, fermented fruit drink, fermented soymilk drink and the like can be mentioned. “Fermented milk” refers to a paste or liquid prepared by fermenting milk or dairy product with lactic acid bacteria, bifidobacteria or yeast. Therefore, the fermented milk contains a form of yoghurt as well as a drink form. “lactic fermenting beverage” refers to a beverage prepared by firstly fermenting milk or dairy product, as the main raw material, with lactic acid bacteria, bifidobacteria or yeast, to make a paste or liquid, and then diluting it with water.

Examples of other foods and drinks include fermented foods such as pickles, soybean pastes, fermented tea, buns and the like, infant foods such as baby foods and powdered milk, foam preparations, confectionery such as gums, gummies and puddings, noodles, capsules, granules, powders, tablets and the like, dairy products other than the above fermented milk and lactic fermenting beverages, and the like. In particular, since the composition contains an immunostimulant component which retains its function even after heating, the form of a processed food is preferable, for which a heating step is required. In a particularly preferred embodiment, a processed food requiring thermal cooking for hygienic management, for example, nursing care foods and the like can be mentioned. The food and drink of this embodiment is stable even after heating at 75° C. effective for food poisoning prevention, and is provided as an immunopotentiating agent, particularly an IgA production promotor and an immunostimulant.

In addition, the food and drink of the present embodiment includes functional foods such as foods for specified health use, health foods and the like, which are based on the concept of prevention of infection, prevention of diarrhea and the like, and indicated as needed. The term, “health food” means a food which is more aggressive than ordinary food and intended for the purpose of health, health maintenance, health promotion, etc. For example, a liquid or semisolid, solid product, specifically, cookies, snacks, jellies, candies, yoghurts, sweet buns etc. confectionery, soft drink, nutritious drink, soup, and the like.

### (Pharmaceutical Product)

When the composition of the present embodiment is used as a pharmaceutical product, a suitable pharmaceutically acceptable carrier can be used together with cells of strain 10H to prepare and apply in the form of a general pharmaceutical composition. As the pharmaceutical carrier, a diluent or excipient such as a filler, an extender, a binder, a moisturizer, a disintegrant, a surface-active agent, a lubricant and the like, which are known to be used in this field, are illustrated. These are appropriately selected and used depending on the unit dosage form of the preparation to be obtained.

As a unit dosage form of the pharmaceutical composition, various forms can be employed, but an oral administration preparation form and an external administration preparation form are preferable. Representative oral administration preparation includes a tablet, pill, powder, solution, suspension, emulsion, granule and capsule.

In molding the form of the tablet, the following pharmaceutical carriers can be used, for example, excipients such as lactose, white sugar, sodium chloride, glucose, urea, starch, calcium carbonate, kaolin, crystalline cellulose, silicic acid, potassium phosphate and the like; binders such as water, propanol, simple syrup, dextrose solution, starch solution, gelatin solution, carboxymethylcellulose, hydroxypropyl cellulose, methyl cellulose, polyvinylpyrrolidone and the like; disintegrating agents such as carboxymethyl cellulose sodium salt, carboxymethyl cellulose calcium salt, low-substituted hydroxypropyl cellulose, dry starch, sodium alginate, agar powder, powdery laminaran, hydrogen carbonate, sodium bicarbonate, calcium carbonate and the like; surfactants such as polyoxyethylene sorbitan fatty acid esters, sodium lauryl sulfate, stearic acid monoglyceride and the like; disintegration inhibitors such as white sugar, stearin, cocoa butter, hydrogenated oil and the like; absorption promotors such as quaternary ammonium base, sodium lauryl sulfate and the like; moisturizers such as glycerin, starch and the like; adsorbents such as starch, lactose, kaolin, bentonite, colloidal silicic acid and the like; lubricants such as purified talc, stearate, borate powder, polyethylene glycol and the like. Further, the tablets may be formulated to have ordinary coatings as needed, such as sugar-coated tablets, gelatin-coated tablets, enteric coated tablets, film-coated tablets or double-layered tablets, multilayered tablets.

In molding the form of the pill, the following pharmaceutical carrier can be used, for example, excipients such as glucose, lactose, starch, cacao butter, hydrogenated vegetable oil, kaolin, talc and the like; binders such as gum arabic powder, powdered tragacanth, gelatin, disintegrating agents such as laminaran and agar and the like.

Furthermore, coloring agents, preservatives, aromatic agents, flavoring agents, sweetening agents, etc. and other medicinal products may be contained in the pharmaceutical composition, if necessary.

The method for administering the pharmaceutical composition is not particularly limited and is determined depending on various formulations, age, sex and other conditions of the patient, degree of disease, and the like. The dosage is appropriately selected depending on the usage, the patient's age, sex and other conditions, the degree of disease, and the like. Usually, the amount of cells of strain 10H, which is the active ingredient, is about 0.5 to about 20 mg, and the formulation can be administered to humans in 1 to 4 divided doses a day.

### (External Preparation)

When the composition of the present embodiment is prepared as an external preparation such as a cosmetic, an external medicine, a quasi-drug and the like, an appropriate pharmaceutically acceptable carrier is used together with cells of strain 10H, to prepare a general form of external composition for practical use.

Examples of such a pharmaceutical carrier include moisturizers such as glycerin, petroleum jelly, urea, hyaluronic acid, heparin and the like; ultraviolet absorbing or scattering agents such as PABA derivatives (paraaminobenzoic acid, ESCALOL 507 etc.), cinnamic acid derivatives (Neo Heliopan, PARSOL MCX, SUNGUARD B, etc.), salicylic acid derivatives (octyl salicylate and the like), benzophenone derivatives (ASL-24, ASL-24S etc.), dibenzoylmethane derivatives (PARSOL A, PARSOL DAM, etc.), heterocyclic derivatives (TINUVIN series, etc.), titanium oxide and the like; metal scavengers such as disodium edetate, trisodium edetate, citric acid, sodium citrate, tartaric acid, sodium tartarate, lactic acid, malic acid, sodium polyphosphate, sodium metaphosphate, gluconic acid and the like; sebum inhibitors such as salicylic acid, sulfur, caffeine and tannin; antiseptic disinfectants such as benzalkonium chloride, benzethonium chloride and chlorohexidine gluconate; anti-inflammatories such as diphenhydramine chloride, tranexamic acid, guaiazulene, azulene, allantoin, Hinokitiol, glycyrrhizic acid and its salts, glycyrrhizic acid derivatives and glycylrrhetinic acid; vitamins such as vitamin A, vitamin Bs (B1, B2, B6, B12, B15), folic acid, nicotinic acids, pantothenic acids, biotin, vitamin C, vitamin Ds (D2, D3), vitamin E, ubiquinone and vitamin Ks (K1, K2, K3, K4); amino acids and derivatives thereof, such as aspartic acid, glutamic acid, alanine, lysine, glycine, glutamine, serine, cysteine, cystine, tyrosine, proline, arginine and pyrrolidonecarboxylic acid; skin-lightening agents such as retinol, tocopherol acetate, magnesium ascorbate phosphate, ascorbic acid glucoside, arbutin, kojic acid, ellagic acid and placenta extract; antioxidants such as butylhydroxytoluene, butylated hydroxyanisole and propyl gallate; astringent agents such as zinc chloride, zinc sulfate, zinc phenate, zinc oxide and aluminum potassium sulfate; sugars such as glucose, fructose, maltose, sucrose, trehalose, erythritol, mannitol, xylitol and lactitol; various plant extracts such as licorice, chamomile, horse chestnut, strawberry geranium, paeoniae radix, quince, scutellaria, phellodendron bark, coptis rhizome, Houttuyniae herba and Ginkgo biloba; and in addition, oil ingredients, surfactants, thickeners, alcohols, powder ingredients, colors, and the like.

Specific examples of the external preparation include cosmetic creams, emulsion, lotion, packing agent, skin milk (emulsion), gel agent, powder, lip cream, lipstick, under make-up, foundation, sun care, bath preparation, body shampoo, body rinse, soap, cleansing foam, ointment, patch, jelly, aerosol and the like.

### (Feed)

In the case where the composition of the present embodiment is used as a feed, the preparation for oral administration (aqueous solution, emulsion, granule, powder, capsule, tablet or the like) is enumerated, for example, for the prevention of infectious diseases at the non-antibiotic administration period of chickens, and at the weaning stage of pigs, cattle and the like.

### (IV) Process for Producing the Immunostimulant

In another embodiment of the present invention, there is provided a process for producing an immunostimulant, that is a composition for stimulating immunity of a subject in need thereof. The process comprises the steps of inoculating the isolated bacterial strain of Lactobacillus kosoi 10H deposited under the accession number NITE BP-02811 in a culture medium of high sugar concentration, wherein the sugar includes D-fructose, and growing cells of the bacterial strain in the culture medium. In the present embodiment, the phrase, “a culture medium of high sugar concentration” means that the medium contains one or plurality of sugars in high concentration, and the high sugar concentration can be adjusted by, for example, adding a sugar solution that includes monosaccharides such as glucose, fructose, mannose and the like; disaccharides such as sucrose, lactose and the like; oligosaccharides, polysaccharides, sugar alcohols or the like. The medium of high sugar concentration is a medium having an osmotic pressure higher than that of usual culture medium. The medium of high sugar concentration, for example, has a final concentration of the saccharides to be added of at least 10% by mass, preferably 10 to 40% by mass, and strain 10H can grow even at a sugar concentration of 40% by mass or more. Furthermore, the medium contains D-fructose as the carbon source of strain 10H in an amount of 5 to 20% by mass, preferably 5 to 10% by mass. D-fructose may be supplied not only as a monosaccharide but also as a medium component added as sucrose, oligosaccharide or polysaccharide (including vegetables, mushrooms and the like), decomposed during cultivation. Therefore, sucrose containing D-fructose (fruit sugar) as its constituent component and saccharides derived from natural products such as brown sugar or brown sugar are preferable as the medium having high sugar concentration. By culturing in a medium containing such high sugar concentration and D-fructose, strain 10H can be grown as the predominant strain even in the presence of other lactic acid bacterial strains or the like.

Next, the present invention will be described in more detail by giving examples, but the present invention is not limited to these examples at all.

### EXAMPLES

### [Example 1] Isolation of Strain 10H and Culture Conditions

The Lactobacillus kosoi strain 10H was obtained from a vegetable brown sugar fermentation broth produced by ARSOA Saku Factory located in Saku City, Nagano Prefecture, Japan. About 50% (w/w) of sugars were present in this fermentation broth including muscovado (10% w/w), galactooligosaccharides (7% w/w) and brown sugar (33% w/w). A dilution-to-extinction technique (Button et al. (1993) Appl Environ Microbiol 59:881-891; Simu and Hagstrom (2004) Appl Environ Microbiol 70:2445-2451) was employed to isolate the strain, using a sterile 96-well polystyrene microtiter plate (Fukaekasei and Watson).

Lactobacilli MRS broth (Difco Laboratories) having the following composition:

| | | |
|---|---|---|
| Proteose Peptone No. 3 | 10 | g/L |
| Beef extract | 10 | g/L |
| Yeast extract | 5 | g/L |
| Dextrose | 20 | g/L |
| Polysorbate 80 | 1 | g/L |
| Ammonium citrate | 2 | g/L |
| Sodium acetate | 5 | g/L |
| Magnesium sulfate | 0.1 | g/L |
| Manganese sulfate | 0.05 | g/L |
| Dipotassium phosphate | 2 | g/L |

was used for 1000 times dilution of the fermentation broth for the starting sample. After two-fold serial dilution (2⁰ to 2¹⁸ times) in the microtiter plate, each well was supplemented with 50% (v/v) sterile fermentation broth. The microtiter plate was placed in an unmodified atmosphere at 30° C. for static culture. After 5 days, a single colony that presented in a well was extracted and subcultured in MRS broth supplemented with 10% (w/v) D-fructose. Lactobacilli MRS agar (Difco Laboratories) supplemented with 10% D-fructose was used to investigate colony formation in a Petri dish. Once the new isolate was identified as a purified strain, it was suspended in glycerol (10%, v/v) and skim milk (10%, v/v) and stored at −80° C.

### [Example 2] Growth Characteristics of Strain 10H

In order to investigate the growth characteristics of strain 10H, cultivation of the strain was performed in only MRS broth, MRS broth supplemented with 5%, 10%, 15% or 20% (w/v) D-fructose, or 0.5% pyruvic acid at 30° C. The absorbance of the culture solution at a wavelength of 660 nm (OD660) was measured using JASCO V-730 UV-Visible spectrophotometer for 0 to 110 hours, and the results are shown in FIG. 1. As shown in FIG. 1, strain 10H did not grow in only MRS broth unless D-fructose was supplemented and it was observed to grow well even when the concentration of D-fructose was increased up to 20%.

### [Example 3] Phylogenetic Analysis

Genomic DNA extraction, PCR-mediated amplification and sequencing of the 16S rRNA gene were carried out by established procedures (Matsuzaki et al. (2014) J Appl Microbiol 116:980-989) using 3130 Genetic Analyzer (Applied Biosystems). The obtained 16S rRNA gene of strain 10H was used as query sequence to search nucleotide databases using the Standard Nucleotide Basic Local Alignment Search Tool (BLAST) program in National Center for Biotechnology Information (NCBI). Based on the homology of the nucleotide sequence of the 16S rRNA gene with the closely related strain, a phylogenetic tree was constructed using the maximum-likelihood method (Felsenstein J (1981) Evolution 35:1229-1242). The results are shown in FIG. 2. A bootstrap value of 50% or more is shown at a branch point. Also, the accession number of GenBank is shown in parentheses.

The draft genome sequence of strain 10H was analyzed using the Ion Torrent PGM system in a conventional manner, and determined to be an average G+C content of 30.5%. 67 contigs were assembled, in which 1376 CDS, one tmRNA, three rRNA genes, 56 tRNA genes and one repeat region were annotated. Average Nucleotide Identity (ANI) between strain 10H and three type strains, L. kunkeei YH-15, L. ozensis Mizu2-1 and L. apinorum Fhon13N, was estimated for each using OrthoANIu (Yoon et al. (2017) Antonie Van Leeuwenhoek 110:1281-1286) employing genomic sequences retrieved from the NCBI. It has been reported that pheS and rpoA partial gene sequences provide an alternative tool to 16S rRNA gene sequences for the identification of different species of the genus Lactobacillus (Naser et al. (2007) Int J Syst Evol Microbiol 57:2777-2789 2007). The genomic sequences of the pheS and rpoA genes of L. kunkeei YH-15 and L. apinorum Fhon13N were retrieved from the NCBI, and the sequence similarities between that and of strain 10H were respectively compared. The results are shown in Table 1 below.

TABLE 1

| | | L. kunkeei | L. apinorum |
|---|---|---|---|
| | Strain 10H | YH-15 | Fhon13 |
| Strain 10H | — | 79.44% (pheS) | 81.46% (pheS) |
| L. kunkeei YH-15 | 89.17% (rpoA) | — | 84.73% (pheS) |
| L. apinorum Fhon13 | 89.91% (rpoA) | 94.85% (rpoA) | — |

From these results, it was found that the sequences of the pheS gene of strain 10H showed similarities of 79.4 and 81.5% to L. kunkeei YH-15 and L. apinorum Fhon13N, respectively, while the similarity between L. kunkeei YH-15 and L. apinorum Fhon13N was 84.7%. The similarity in the rpoA gene between L. kunkeei YH-15 and L. apinorum Fhon13N was 94.8%, higher than the similarities of strain 10H to L. kunkeei YH-15 (89.2%) and to L. apinorum Fhon13N (89.9%). The ANI value of strain 10H and L. kunkeei YH-15 was calculated to be 74.46%, whereas the ANI value between strain 10H and L. apinorum Fhon13N was 75.26% and between strain 10H and L. ozensis Mizu2-1 was 73.31%. Since these values are below the ANI threshold range (95-96%) for species demarcation (Lee et al. 2016), strain 10H is concluded to belong to a species different from L. kunkeei, L. apinorum, and L. ozensis.

### [Example 4] Measurement of IgA Production-Inducing Activity of Test Bacterial Sample

### (Preparation of Peyer's Patch Cells)

Male BALB/cA mice at 6 weeks of age (purchased from CREA Japan) were bred for 1 week with AIN-76A DIET (purchased from Research Diets), then euthanized with carbon dioxide gas, and Peyer's patches were collected. The Peyer's patch cells were washed with RPMI10 medium [RPMI1640 medium (Gibco BRL) with 100 U/mL penicillin, 100 μg/mL streptomycin, 55 μmol/L 2-mercaptoethanol and 10% fetal bovine serum (FBS; Gibco BRL)], 25 mmol/1 HEPES, 5 mmol/L EDTA (pH 8.0) and 1 mmol/L dithiothreitol for 45 minutes at 37° C. under 5% CO₂ condition. After washing again with RPMI10 medium, the cells were cultured in RPMI10 medium supplemented with 400 U/mL type I collagenase (Sigma) and 30 U/mL DNase I (Takara) for 50 minutes at 37° C. and 5% CO₂. The reaction solution was filtered through a 45 μm filter, replaced with RPMI10 medium, and the cell number was adjusted to 5.0×10⁶ cells/ml.

(Preparation of Test Bacterial Solution)

The test bacteria were statically cultured overnight at 30° C. in MRS medium (Difco Laboratories), and after replacement with physiological saline, the absorbance (OD600) was adjusted to 0.02. Heat treatment was carried out at 70° C. or 100° C. for 30 minutes as required. Lactobacillus kosoi sp. nov. Laboratory custody was used. Bifidobacterium catenulatum (JCM1194), Bifidobacterium breve (JCM1192), Bifidobacterium animalis subsp. lactis (JCM10602), Bifidobacterium adolescentis (JCM1275), Bifidobacterium pseudolongum subsp. pseudolongum (JCM1205), Bifidobacterium pseudocatenulatum (JCM1200), Bifidobacterium longum subsp. longum (JCM1217), Bifidobacterium longum subsp. infantis (JCM1222), Lactobacillus plantarum subsp. plantarum (JCM1149), Lactobacillus johnsonii (JCM2012), Lactobacillus casei (JCM1134), Lactobacillus reuteri (JCM1112), Lactobacillus gasseri (JCM1131), Lactococcus lactis subsp. lactis (JCM5805) was purchased from RIKEN Japan Collection of Microorganisms (JCM). Lactobacillus rhamnosus GG (ATCC53103) was purchased from American Type Culture Collection (ATCC).

(Measurement of IgA)

100 μl of the Peyer's patch cells prepared and 100 μl of the test bacterial solution were co-cultured in a 96-well T-cell activation plate (Becton Dickinson) for 5 days at 37° C. under 5% CO₂. After centrifugation, the amount of IgA in the obtained culture supernatant was measured with a mouse IgA ELISA kit (Bethyl Laboratories).

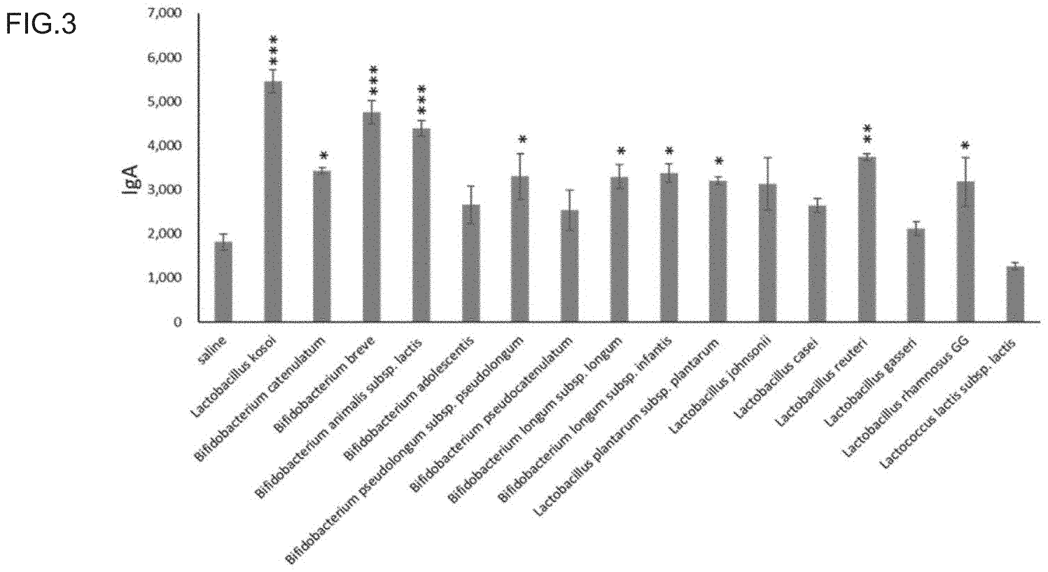

The results are shown in Table 2 and FIG. 3. The strain 10H showed a very high IgA production inducing activity, which was higher than any of the other 15 test bacteria used in this comparison.

TABLE 2
Amount of IgA induced by bacterial cells (ng/ml)

| | | | | | Bifidobacterium |
|---|---|---|---|---|---|
| | | | Bifidobacterium | Bifidobacterium | animalis subsp. |
| | saline | Lactobacillus kosoi | catenulatum | breve | lactis |
| ng/ml | 1814 | 5462 | 3426 | 4759 | 4394 |
| Standard Deviation (SD) | 312 | 451 | 117 | 467 | 295 |
| Standard Error (SE) | 180 | 261 | 68 | 270 | 170 |
| | | Bifidobacterium | | | |
| | | pseudolongum | | Bifidobacterium | Bifidobacterium |
| | Bifidobacterium | subsp. | Bifidobacterium | longum subsp. | longum subsp. |
| | adolescentis | pseudolongum | pseudocatenulatum | longum | infantis |
| ng/ml | 2653 | 3303 | 2541 | 3297 | 3372 |
| Standard Deviation (SD) | 752 | 888 | 781 | 479 | 367 |
| Standard Error (SE) | 434 | 513 | 451 | 276 | 212 |
| | Lactobacillus | | | | |
| | plantarum | | | | |
| | subsp. | Lactobacillus | Lactobacillus | Lactobacillus | Lactobacillus |
| | plantarum | johnsonii | casei | reuteri | gasseri |
| ng/ml | 3205 | 3133 | 2649 | 3737 | 2112 |
| Standard Deviation (SD) | 158 | 1027 | 274 | 138 | 275 |
| Standard Error (SE) | 91 | 593 | 158 | 80 | 159 |
| | Lactobacillus | Lactococcus lactis | | | |
| | rhamnosus GG | subsp. lactis | | | |
| ng/ml | 3179 | 1261 | | | |
| Standard Deviation (SD) | 947 | 137 | | | |
| Standard Error (SE) | 547 | 79 | | | |

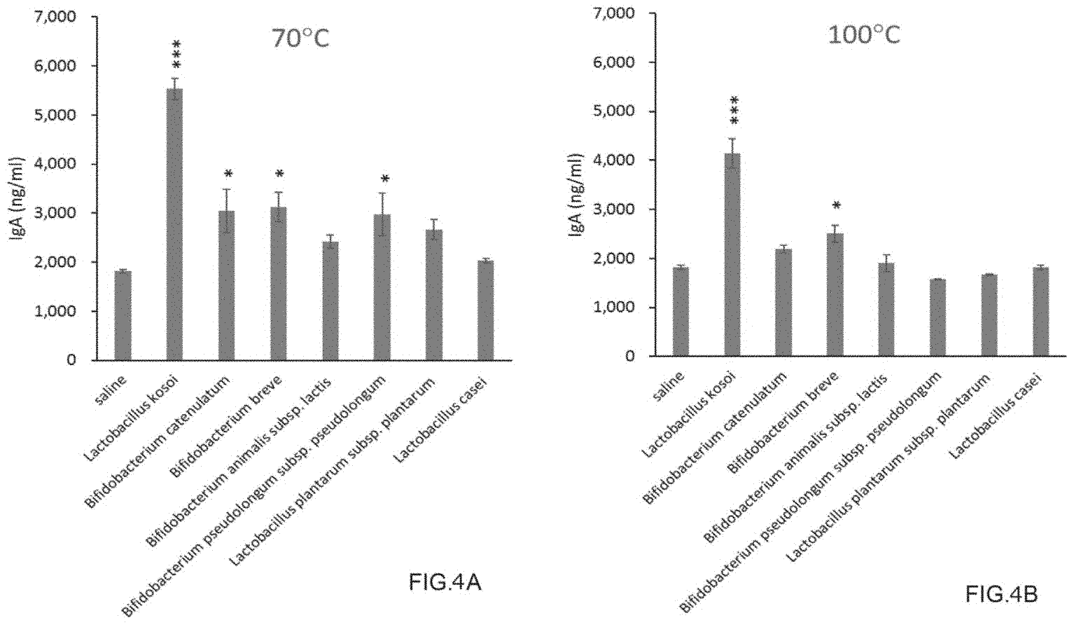

Subsequently, the same test was carried out using the test bacterial samples subjected to heat-treatment at 70° C. for 30 minutes, and the results are shown in the following Table 3 and FIG. 4A.

TABLE 3
Amount of IgA induced by heat treated bacterial cells at 70° C. (ng/ml)

| | | Lactobacillus | Bifidobacterium | Bifidobacterium |
|---|---|---|---|---|
| | saline | kosoi | catenulatum | breve |
| ng/ml | 1814 | 5529 | 3042 | 3121 |
| Standard Deviation (SD) | 77 | 368 | 757 | 525 |
| Standard Error (SE) | 44 | 213 | 437 | 303 |
| | | Bifidobacterium | Lactobacillus | |
| | Bifidobacterium | pseudolongum | plantarum | |
| | animalis subsp. | subsp. | subsp. | Lactobacillus |
| | lactis | pseudolongum | plantarum | casei |
| ng/ml | 2425 | 2971 | 2663 | 2034 |
| Standard Deviation (SD) | 228 | 749 | 346 | 81 |
| Standard Error (SE) | 132 | 432 | 199 | 47 |

In addition, the same test was conducted using the test samples subjected to heat treatment at 100° C. for 30 minutes, and the results are shown in the following Table 4 and FIG. 4B. In the significance test, saline was used as a control group, and comparison between groups with the control group was performed by Dunnett's multiple comparison test. The significance level at less than 5% risk rate indicates as *, less than 1% risk rate as **, and less than 0.1% risk rate as ***.

TABLE 4
Amount of IgA induced by heat treated bacterial cells at 100° C. (ng/ml)

| | | Lactobacillus | Bifidobacterium | Bifidobacterium |
|---|---|---|---|---|
| | saline | kosoi | catenulatum | breve |
| ng/ml | 1814 | 4143 | 2191 | 2504 |
| Standard Deviation (SD) | 77 | 513 | 130 | 302 |
| Standard Error (SE) | 44 | 296 | 75 | 174 |
| | | Bifidobacterium | Lactobacillus | |
| | Bifidobacterium | pseudolongum | plantarum | |
| | animalis subsp. | subsp. | subsp. | Lactobacillus |
| | lactis | pseudolongum | plantarum | casei |
| ng/ml | 1904 | 1576 | 1670 | 1803 |
| Standard Deviation (SD) | 291 | 18 | 15 | 77 |
| Standard Error (SE) | 168 | 10 | 9 | 40 |

From these results, strain 10H did not show a decrease in immunity-inducing activity by heating at 70° C. for 30 minutes. Approximately 75% of the activity was retained even by heating at 100° C. for 30 minutes. The residual activities at both 70° C. and 100° C. heat-treatments were higher than those of any other bacteria used in this comparison. Lactobacillus kosoi strain 10H is expected to be a highly heat-resistant immunostimulant.

What is claimed is:

1. An isolated bacterial strain of Lactobacillus kosoi 10H deposited under the accession number NITE BP-02811.

2. The isolated bacterial strain according to claim 1, comprising nucleotide sequences of SEQ ID Nos. 1, 3, 5 and 7.

3. A method for stimulating immunity of a subject in need thereof, comprising administering to the subject a composition comprising an effective amount of cells of Lactobacillus kosoi 10H deposited under the accession number NITE BP-02811.

4. The method according to claim 3, wherein the immunity is stimulated by promoting IgA production in mucosa of the subject.

5. The method according to claim 4, wherein the mucosa is intestinal mucosa.

6. The method according to claim 3, wherein the cells are heat-treated at 70° C. for one minute or more.

7. The method according to claim 3, wherein the subject is a mammal.

8. The method according to claim 3, wherein the composition is in a form of a food and drink, a pharmaceutical composition, an external preparation or a feed.

9. The method according to claim 4, wherein the composition is in a form of a food and drink, a pharmaceutical composition, an external preparation or a feed.

10. The method according to claim 6, wherein the composition is in a form of a food and drink, a pharmaceutical composition, an external preparation or a feed.

11. A composition for stimulating immunity of a subject in need thereof, the composition comprising cells of the isolated bacterial strain according to claim 1, as an active ingredient.

12. The composition according to claim 11, wherein the immunity is stimulated by promoting IgA production in mucosa of the subject.

13. The composition according to claim 11, wherein the cells are heat-treated at 70° C. for one minute or more.

14. The composition according to claim 11, which is in a form of a food and drink, a pharmaceutical composition, an external preparation or a feed.

15. A process for producing a composition for stimulating immunity of a subject in need thereof, the process comprising the steps of:
inoculating the isolated bacterial strain according to claim 1 in a culture medium of high sugar concentration, wherein the sugar includes D-fructose, and
growing cells of the bacterial strain in the culture medium.

16. The process according to claim 15, further comprising a step of heat-treating the cells at 70° C. for one minute or more.
